(19) United States
(12) Patent Application Publication
(10) Pub. No.: US 2002/0151462 A1
(43) Pub. Date: Oct. 17, 2002

(54) Helicobacter pylori membrane proteins

(76) Inventor: Ling Lissolo, Marcy L'Etoile (FR)

Correspondence Address: Paul T Clark, Clark and Elbing LLP, 176 Federal Street, Boston, MA, 02110, US

(21) Appl. No.: 09/488,737

(22) Filed: Jan. 20, 2000

Related U.S. Application Data
(63) Continuation of application No. 08/849,627, filed on Feb. 19, 1998, now abandoned.
Application No. 08/849,627 is a 371 of international application No. PCT/FR96/01552, filed on Oct. 4, 1996.

(51) Int. Cl. A01N 37/18; A61K 38/00; A61K 31/545

(52) U.S. Cl. 514/2

(57) ABSTRACT

The invention relates to proteins of Helicobacter pylori, in a substantially purified forms, that can be obtained from a membrane fraction of H. pylori and of which the molecular weights after electrophoresis on a 10% polyacrylamide gel in the presence of SDS are approximately 54, 50, 32-35, or 30 kDa.

The object of the present invention is Helicobacter pylori proteins newly obtained in substantially purified form, as well as the pharmaceutical compositions containing them.

Helicobacter is a bacterial genus characterized by Gram-negative helical bacteria. Several species colonize the gastrointestinal tract of mammals. There may be mentioned in particular H. pylori, H. heilmanii, H. felis and H. mustelae. Although H. pylori is the species most commonly associated with human infections, in some admittedly rare cases, it has been possible to isolate in man H. heilmanii and H. felis.

Helicobacter infects more than 50% of the adult population in developed countries and nearly 100% of that of developing countries, thereby making it one of the predominant infectious agents worldwide.

H. pylori is so far exclusively found at the surface of the mucous membrane of the stomach in man and more particularly around the crater lesions of gastric and duodenal ulcers. This bacterium is currently recognized as the aetiological agent of antral gastritis and appears as one of the cofactors required for the development of ulcers. Moreover, it seems that the development of gastric carcinomas may be associated with the presence of H. pylori.

It therefore appears to be highly desirable to develop a vaccine intended to prevent or treat H. pylori infections. Such a vaccine would be most probably of a subunit nature.

Various H. pylori proteins have been characterized or isolated so far. They are especially urease, composed of two subunits A and B of 30 and 67 kDa respectively (Hu & Mobley, Infect. Immun. (1990) 58:992; Dunn et al., J. Biol. Chem. (1990) 265: 9464; Evans et al., Microbial Pathogenesis (1991) 10: 15; Labigne et al., J. Bact, (199) [sic] 173:1920); the vacuole cytotoxin of 87 kDa (VacA) (Cover & Blaser., J. Biol. Chem. (1992) 267:10570; Phadnis et al., Infect. Immun. (1994) 62:1557; WO 93/18150); an immunodominant antigen of 128 kDa associated with the cytotoxin (CagA, also called TagA) (WO 93/18150; U.S. Pat. No. 5,403,924); heat shock proteins HspA and HspB of 13 and 58 kDa respectively (Suerbaum et al., Mol. Microbiol. (1994) 14:959; WO 93/18150); a catalase of 54 kDa (Hazell et al., J. Gen. Microbiol. (1991) 137:57); a fibrillar haemaglutinin (HpaA) of 20 kDa; a histidine-rich protein of 15 kDa (Hpn) (Gilbert et al., Infect. Immun. (1995) 63:2682); an outer membrane protein of 30 kDa (Bölin et al., J. Clin. Microbiol. (1995) 33: 381); a membrane-associated lipoprotein of 20 kDa (Kostrcynska et al., J. Bact. (1994) 176:5938) as well as a family of porins HopA, HopB, HopC and HopD, of molecular weight between 48 and 67 kDa (Exner et al., Infect. Immun. (1995) 63:1567).

Some of these proteins have already been proposed as potential vaccinal antigens. In particular, urease is recognized as being a most preferred antigen which can be used for this purpose (WO 94/9823; WO 95/3824; WO 95/22987; Michetti et al., Gastroenterology (1994) 107: 1002). The fact remains that the search for new antigens must continue, especially since it is envisaged that, in order to obtain an optimum vaccinal effect, several antigens will probably have to be incorporated into a vaccine.

In summary, it still appears necessary to identify additional antigens in order to incorporate them into a vaccine of high efficacy.

Accordingly, the subject of the invention is especially an H. pylori protein in a substantially purified form, capable of being obtained from an H. pylori membrane fraction, and whose molecular weight after electrophoresis on a 10% polyacrylamide gel in the presence of SDS appears of the order of 54, 50, 32-35 or 30 kDa. When the protein has a molecular weight of about 54 kDa, it is specified, in addition, that it does not react with an anti-catalase antiserum.

An anti-H. pylori catalase antiserum may be especially prepared according to the immunization process described in Example 5 below, using a catalase preparation obtained by chromatography, as described in Example 6.

“Substantially purified form” is understood to mean that the protein is separated from the medium in which it exists naturally. Among others, it may be a preparation lacking especially the H. pylori cytoplasmic and periplasmic proteins.

The membrane protein whose apparent molecular weight is of the order of 54 kDa is capable of being obtained by a process in which:

(i) the H. pylori bacteria are extracted with 1% n-octyl β-D glucopyranoside, followed by centrifugation;

(ii) a bacterial pellet is recovered and it is treated with lysozyme and subjected to sonication, followed by centrifugation;

(iii) a centrifugation pellet is recovered and it is subjected to washing with 20 mM Tris-HCl buffer pH 7.5, followed by centrifugation;

(iv) the membrane fraction consisting of the centrifugation pellet is recovered and it is resuspended in aqueous medium, advantageously in carbonate buffer pH 9.5 containing 5% zwittergent 3-14;

(v) the membrane fraction is subjected to an anion-exchange chromatography on a Q-Sepharose column in a 0-0.5 M NaCl gradient, advantageously in a carbonate buffer pH 9.5 containing 0.1% zwittergent 3-14, followed by washing in 1 M NaCl, advantageously in a carbonate buffer pH 9.5 containing 0.1% zwittergent 3-14;

(vi) the fraction eluted at the start of washing in 1 M NaCl is recovered and it is subjected to an anion-exchange chromatography on a DEAE-Sepharose column, in a 0-0.5 M NaCl gradient, advantageously in Tris-HCl buffer pH 7.5 containing 0.1% zwittergent 3-14 (advantageously, the fraction in 1 M NaCl is first dialysed against Tris-HCl buffer pH 7.5 containing 0.1% zwittergent 3-14); and

(vii) the fraction eluted in 0.1-0.25 M NaCl is recovered.

The membrane protein whose apparent molecular weight is of the order of 50 kDa is capable of being obtained by a process in which:

(i) the H. pylori bacteria are extracted with 1% n-octyl β-D glucopyranoside, followed by centrifugation;

(ii) a bacterial pellet is recovered and it is treated with lysozyme and subjected to sonication, followed by centrifugation;

(iii) a centrifugation pellet is recovered and it is subjected to washing with 20 mM Tris-HCl buffer pH 7.5, followed by centrifugation;

(iv) the membrane fraction consisting of the centrifugation pellet is recovered and it is resuspended in aqueous medium, advantageously in carbonate buffer pH 9.5 containing 5% zwittergent 3-14;

(v) the membrane fraction is subjected to an anion-exchange chromatography on a Q-Sepharose column in a 0-0.5 M NaCl gradient, advantageously in a carbonate buffer pH 9.5 containing 0.1% zwittergent 3-14, followed by washing in 1 M NaCl, advantageously in a carbonate buffer pH 9.5 containing 0.1% zwittergent 3-14;

(vi) the fraction eluted at the start of washing in 1 M NaCl is recovered and it is subjected to an anion-exchange chromatography on a DEAE-Sepharose column, in a 0-0.5 M NaCl gradient, advantageously in Tris-HCl buffer pH 7.5 containing 0.1% zwittergent 3-14 (advantageously, the fraction in 1 M NaCl is first dialysed against Tris-HCl buffer pH 7.5 containing 0.1% zwittergent 3-14); and

(vii) the fraction eluted in 0.3-0.4 M NaCl is recovered.

The membrane protein whose apparent molecular weight is of the order of 30 kDa. is capable of being obtained by a process in which:

(i) the H. pylori bacteria are extracted with 1% n-octyl β-D glucopyranoside, followed by centrifugation;

(ii) a bacterial pellet is recovered and it is treated with lysozyme and subjected to sonication, followed by centrifugation;

(iii) a centrifugation pellet is recovered and it is subjected to washing with 20 mM Tris-HCl buffer pH 7.5, followed by centrifugation;

(iv) the membrane fraction consisting of the centrifugation pellet is recovered and it is resuspended in aqueous medium, advantageously in carbonate buffer pH 9.5 containing 5% zwittergent 3-14;

(v) the membrane fraction is subjected to an anion-exchange chromatography on a Q-Sepharose column in a 0-0.5 M NaCl gradient, advantageously in a carbonate buffer pH 9.5 containing 0.1% zwittergent 3-14;

(vi) the fraction eluted in 0.28-0.35 M NaCl is recovered and it is subjected to an anion-exchange chromatography on a DEAE-Sepharose column, in a 0-0.5 M NaCl gradient, advantageously in Tris-HCl buffer. pH 7.5 containing 0.1% zwittergent 3-14 (advantageously, the fraction in 1 M NaCl is first dialysed against Tris-HCl buffer pH 7.5 containing 0.1% zwittergent 3-14); and

(vii) the fraction corresponding to the direct eluate is recovered (absence of NaCl).

The membrane protein whose apparent molecular weight is of the order of 32-35 kDa is capable of being obtained by a process in which:

(i) the H. pylori bacteria are extracted with 1% n-octyl β-D glucopyranoside, followed by centrifugation;

(ii) a bacterial pellet is recovered and it is treated with lysozyme and subjected to sonication, followed by centrifugation;

(iii) a centrifugation pellet is recovered and it is subjected to washing with 20 mM Tris-HCl buffer pH 7.5, followed by centrifugation;

(iv) the membrane fraction consisting of the centrifugation pellet is recovered and it is resuspended in aqueous medium, advantageously in carbonate buffer pH 9.5;

(v) the suspension obtained in (iv) is centrifuged at about 200,000×g and the supernatant is recovered;

(vi) the pH of the supernatant obtained in (v) is reduced to about pH 7, advantageously by dialysing against phosphate buffer pH 7;

(vii) the preparation obtained in (vi) is subjected to a cation-exchange chromatography on an SP-Sepharose column in a 0-0.5 M NaCl gradient, advantageously in a phosphate buffer pH 7; and

(vii) the fraction eluted in 0.26-0.31 M NaCl is recovered.

The 54, 50, 32 and 30 kDa proteins according to the invention are probably intrinsic membrane proteins or proteins associated with the membrane. The 54 kDa protein does not react with anti-catalase antibodies, nor in Western blotting, or in dot blotting. The 30 kDa protein does not react with anti-urease A subunit antibodies, nor in Western blotting, or in dot blotting. The 32 kDa protein proves to be an alkaline protein; its molecular weight may appear slightly greater e.g. of the order of 35 kDa under certain experimental conditions.

The N-terminal sequence of the 50 kDa protein of an H. pylori strain (ATCC 43579) is as follows (one-letter code): MKEKFNRTKPHVNIGTIGHVDH. This information does not exclude the fact that equivalent proteins capable of being purified according to the process indicated above can have a slightly different N-terminal sequence, since they may be derived from another bacterial strain. Such a difference would indeed reflect the phenomenon of allelic variance commonly encountered within the same species. For example, a bacterial species is usually represented by a group of strains which differ from each other in minor allelic characteristics. A polypeptide which fulfils the same biological function in different strains may have an amino acid sequence which is not different for all the strains. Such an allelic variation also exists in DNA.

The allelic differences at the level of the amino acid sequence may consist of one or more substitutions, deletions or additions of amino acids, which do not alter the biological function.

“Biological function” is understood to mean the function of the protein which participates in the survival of the cells in which the protein exists naturally (even if the function is not absolutely essential). For example, the function of a porin is to allow compounds present in the external medium to enter inside the cell. The biological function is distinct from the antigenic function. A protein may have more than one biological function.

The subject of the invention is also a protein in a substantially purified form and which may have been purified according to one of the processes described above from a bacterium of the genus Helicobacter, e.g. H. pylori, H. heilmanii, H. felis and H. mustelae.

The subject of the invention is also any protein or polypeptide, in a substantially purified form, insofar as it is analogous, in terms of antigenicity, to a Helicobacter protein capable of being purified according to one of the processes described above. As regards the polypeptides, they are especially polypeptides derived by fragmentation or by mutation of one or more amino acids, e.g. by deletion, addition or substitution, of a protein which exists in nature and whose purified form may be obtained according to one of the processes described above. Such polypeptides may be especially obtained by enzymatic digestion with the aid of proteases such as pepsin or trypsin. It is not necessary for such polypeptides to be purified according to one of the processes described above.

The present description uses the terms “protein” and “polypeptide” independently of the size of the molecules (length of the amino acid chain) and of the possible post-translational modifications. In the remainder of the description, the term “polypeptide” is reserved to designate a product derived from a protein by fragmentation or mutation.

A protein or a polypeptide according to the invention should be capable of being recognized by monospecific antibodies raised against a Helicobacter protein capable of being purified according to one of the processes described above. This specific antigenicity may be revealed according to a number of methods; for example by Western blotting (Towbin et al., PNAS (1979) 76:4350), dot blotting-and ELISA.

In Western blotting, the product intended to be tested, e.g. either in the form of a purified preparation or in the form of a bacterial extract, is subjected to an SDS-Page gel electrophoresis (10% polyacrylamide) as described by Laemmli U.K., Nature (1970) 227:680. After transferring onto a nitrocellulose membrane, the latter is incubated with a monospecific hyperimmune serum diluted in the range of dilutions from 1:50 to 1:5000, preferably from 1:100 to 1:500. The specific antigenicity is demonstrated as soon as a band corresponding to the test product exhibits a reactivity at one of the dilutions included in the range established above.

In ELISA, the product intended to be tested is preferably used to coat wells. A purified preparation is preferably used although a total extract can also be used. Briefly, 100 μl of a preparation containing 10 μg of protein/ml are distributed into the wells of a 96-well plate. The plate is incubated for 2 h at 37° C. and then overnight at 4° C. The plate is washed with PBS (phosphate buffered saline) buffer containing 0.05% Tween 20 (PBS/Tween buffer). The wells are saturated with 250 μl of PBS containing 1% bovine serum albumin (BSA). The whole is incubated for 1 h at 37° C. and then the plate is washed with PBS/Tween buffer. A monospecific rabbit antiserum is serially diluted in PBS/Tween buffer containing 0.5% BSA. One hundred pi of a dilution are added to each well. The plate is incubated for 90 min at 37° C. and then washed. The plate is visualized according to standard methods. For example, a conjugate peroxidase-goat immunoglobulin against rabbit immunoglobulins is added to the wells. The incubation is continued for 90 min at 37° C. and then the plate is washed. The reaction is developed with the appropriate substrate. The reaction is measured by colorimetry (absorbance measured by spectrophotometry). Under these conditions, a positive reaction is observed when an OD value of 1 is associated with a dilution of at least 1:50, preferably of at least 1:500. The appropriate wavelength at which the optical density is measured depends on the substrate.

In dot blotting, a purified preparation of the product to be tested is preferably used although a total extract can also be used. Briefly, a preparation of the product to be tested containing 100 μg of protein/ml is serially diluted twice in 50 mM Tris-HCl pH 7.5. One hundred μl of each dilution are applied to a 0.45 μm nitrocellulose membrane in a 96-well dot blot apparatus (Biorad). The buffer is removed by placing under vacuum. The wells are washed by addition of 50 mM Tris-HCl pH 7.5 and the membrane is air-dried. The membrane is saturated with blocking buffer (50 mM Tris-HCl pH 7.5, 0.15 M NaCl, 10 g/l skimmed milk) and then incubated with a monospecific antiserum diluted in the range from 1:50 to 1:5000, preferably from 1:50 to 1:500. The reaction is visualized according to standard methods. For example, a conjugate peroxidase-goat immunoglobulin against rabbit immunoglobulins is added to the wells. The incubation is continued for 90 min at 37° C. and then the plate is washed. The reaction is developed with the appropriate substrate. The reaction is measured by colorimetry or chemiluminescence. Under these conditions, a reaction is positive when a color is observed at the level of the spot on the nitrocellulose sheet directly for visualization by colorimetry or on a photographic film for visualization by chemiluminescence, associated with a dilution of at least 1:50, preferably at least 1:500.

According to a specific embodiment, a protein according to the invention may be obtained especially by purification from Helicobacter or expressed by the recombinant route in a heterologous system (which may also be the case for a polypeptide according to the invention). In the latter case, the protein may exhibit post-translational modifications which are not identical to those of the corresponding protein derived from the original strain.

The therapeutic or prophylactic efficacy of a protein or of a polypeptide according to the invention may be evaluated according to standard methods, for example by measuring the induction of a mucosal immune response or the induction of an immune response having a therapeutic or protective effect using e.g. the mouse/H. felis model and the procedures described in Lee et al., Eur. J. Gastroenterology & Hepatology, (1995), 7:303 or Lee et al., J. Infect. Dis. (1995) 172:161, on condition that the following precaution is taken: when the protein is derived from a species other than H. felis, the H. felis strain should be replaced by a Helicobacter strain belonging to the species from which the protein is derived and adapted to this end (the other experimental conditions remaining identical). For example, the capacity of a polypeptide derived by fragmentation from an H. pylori protein to induce a protective or therapeutic effect is tested by substituting an H. pylori strain. Such a strain is proposed by e.g. Kleanthous et al., Abstr. presented at the VIIIth International Workshop on Gastroduodenal Pathology Jul. 7-9 1995 Edinburgh, Scotland. A protective effect is observed once an infection in the gastric tissue is reduced compared with a control group. The infection is evaluated by testing the urease activity, the bacterial load or the leucocyte infiltration. For example, when a reduction in the urease activity in the gastric tissue is observed after a challenge, even if it is not completely abolished it is reasonable to assert that there is partial protection.

Consequently, the invention also relates to (i) a composition of material comprising a protein or a polypeptide according to the invention and a diluent or a carrier; in particular (ii) a pharmaceutical composition intended especially for the prevention or treatment of a Helicobacter infection, which comprises as active ingredient a protein or a polypeptide according to the invention, in a quantity effective from a prophylactic or therapeutic point of view; (iii) the use of a protein or a polypeptide according to the invention as therapeutic or prophylactic agent; (iv) the use of a protein or a polypeptide according to the invention for the manufacture of a medicament intended for the prevention or treatment of a Helicobacter infection; as well as to (v) a method for inducing an immune response against Helicobacter, e.g. H. pylori, H. heilmanii, H. felis and H. mustelae in a mammal, according to which an immunologically effective quantity of a protein or of a polypeptide according to the invention is administered to the said mammal so as to develop an immune response; in particular (vi) a method for the prevention or treatment of a Helicobacterlle [sic] infection, there is administered to an individual a prophylactically or therapeutically effective quantity of a protein or of a polypeptide according to the invention.

The methods and pharmaceutical compositions according to the invention can treat or prevent Helicobacter infections and, consequently, gastrointestinal diseases associated with such infections. They are in particular chronic and atrophic acute gastritis; peptic ulcers, e.g. gastric and duodenal ulcers; gastric cancers; chronic dyspepsias; refractory non-ulcerous dyspepsias; intestinal metaplasias and certain lymphomas (e.g. low grade MALT lymphoma).

A composition according to the invention may be administered by any conventional route in use in the field of vaccines, in particular through a mucosal (e.g. ocular, nasal, oral, gastric, intestinal, rectal, vaginal or the urinary tract) surface or by the parenteral (e.g. subcutaneous, intradermal, intramuscular, intravenous or intraperitoneal) route. The choice of the route of administration depends on a number of parameters such as the adjuvant associated with the protein or the polypeptide according to the invention. For example, if a mucosal adjuvant is used, the nasal or oral route will be preferred. If a lipid formulation is used, the parenteral route, preferably the subcutaneous or intramuscular route, will be chosen.

A composition according to the invention may comprise in addition to a protein or a polypeptide according to the invention, at least one other Helicobacter antigen such as the urease apoenzyme, or a subunit, fragment, homologue, mutant or derivative of this urease.

For use in a composition according to the invention, a protein or a polypeptide according to the invention may be formulated in or with liposomes, preferably neutral or anionic liposomes, microspheres, ISCOMS or virus-like particles (VLPs), so as to promote the targeting of the protein or polypeptide or to enhance the immune response. Persons skilled in the art obtain these compounds without any difficulty; for example see Liposomes: A Practical Approach, RRC New ED, IRL press (1990).

Adjuvants other than liposomes may also be used. A large number are known to persons skilled in the art. Such adjuvants are referenced below:

For parenteral administration, there may be mentioned especially aluminium compounds such as aluminium hydroxide, aluminium phosphate and aluminium hydroxyphosphate. The antigen may be adsorbed or precipitated onto an aluminium compound according to standard methods. Other adjuvants such as RIBI from ImmunoChem (Hamilton, Mont.) may be used for parenteral administration.

For mucosal administration, there may be mentioned especially the bacterial toxins, e.g. the cholera toxin (CT), the heat-labile E. coli toxin (LT), the Clostridium difficile toxin and the pertussis toxin (PT) as well as the detoxified forms (subunit, toxoid or mutant) of these toxins. For example, a preparation containing the B subunit of CT (CTB) and a smaller quantity of CT may be used. Fragments, homologues and derivatives of these toxins are likewise appropriate insofar as they retain an adjuvant activity. Preferably, a mutant having a reduced toxicity is used. Such mutants are described e.g. in WO 95/17211 (Arg-7-Lys CT mutant), WO 95/34323 (Arg-9-Lys Glu-129-Gly PT mutant) and WO 96/6627 (Arg-192-Gly LT mutant). Other adjuvants, such as the major bacterial lipopolysaccharide (MPLA) of e.g. E. coli, Salmonella Minnesota, Salmonella typhimurium or Shigella flexneri, may be used for mucosal administration.

Adjuvants useful both for mucosal and parenteral administration include especially polyphosphazine (WO 95/2415), DC-chol (3-beta-[N-U.S. Pat. No. (N′,N′-dimethylaminomethane)carbamoyl]cholesterol (USP 5,283,185 and WO 96/14831) and QS-21 (WO 88/9336).

The administration may be made as a single dose or as a dose repeated once or several times after a certain period. The appropriate dosage varies according to various parameters, for example the individual treated (adult or child), the vaccinal antigen itself, the mode and frequency of administration, the presence or absence of adjuvant and if present, the type of adjuvant and the desired effect (e.g. protection or treatment), as can be determined by persons skilled in the art. In general, an antigen according to the invention may be administered in a quantity ranging from 10 μg to 500 mg, preferably from 1 mg to 200 mg. In particular, it is indicated that a parenteral dose should not exceed 1 mg, preferably 100 μg. Higher doses may be prescribed for e.g. oral use. Independently of the formulation, the quantity of protein administered to man by the oral route is for example of the order of 1 to 10 mg per dose, and at least 3 doses are recommended at 4-week intervals.

A composition according to the invention may be manufactured in a conventional manner. In particular, a protein or a polypeptide according to the invention is combined with a diluent or a carrier which is pharmaceutically acceptable, e.g. water or a saline solution such as a phosphate buffered saline (PBS), optionally supplemented with a bicarbonate salt such as sodium bicarbonate, e.g. 0.1 to 0.5 M when the composition is intended for oral or intragastric administration. In general, the diluent or carrier is selected on the basis of the mode and route of administration and of standard pharmaceutical practices. Diluents and carriers which are pharmaceutically acceptable, as well as all that is necessary for their use in pharmaceutical formulations are described in Remington's Pharmaceutical Sciences, a standard reference text in this field, and in USP/NP.

In a more detailed manner, it is proposed, by way of example, to administer a protein or a polypeptide according to the invention by the oral route. To this end, a protein or a polypeptide according to the invention may be encapsulated alone or in the presence of other H. pylori proteins in gelatin capsules in order to protect the antigen against degradation by the gastric juice, or administered in the presence of sodium bicarbonate. Such formulations have already been used for pharmaceutical compositions (Black et al., Dev. Biol. Stand. (1983), 53:9). The protein may also be encapsulated in PLGA microspheres (glycolic acid and lactic acid copolymers) according to the procedure described elsewhere (Eldridge et al., Curr. Top. Microbiol. Immuno. (1989) 146:59); the protein may also be encapsulated in liposomes prepared according to widely-described conventional methods (“Liposomes: a practical approach, Ed. RRC New, D. Rickwood & B. D. Hames, 1990, Oxford University Press, ISBN 0-19-963077-1).

Alternatively, a protein or a polypeptide according to the invention may be administered by the parenteral route. To do this, a protein or a polypeptide according to the invention is adsorbed onto alumina gel in a completely conventional manner. The protein in solution at 1 mg/ml in a buffer whose pH is close to 6.5 is brought into contact, for 1 hour, with aluminium hydroxide at 10 mg/ml, measured at AL⁺⁺⁺. The final composition of the preparation is the following: protein 50 μg/ml, AL⁺⁺⁺250 μg/ml, merthiolate 1/10,000, the whole in PBS. As in the case of oral administration, 3 injections are recommended, each at an interval of 4 weeks from the preceding one.

A polypeptide according to the invention may also be useful as diagnostic reagent, for example for detecting the presence of anti-Helicobacter antibody in a biological sample, e.g. a blood sample. To this end, such a polypeptide advantageously comprises 5 to 80 amino acids, preferably 10 to 50 amino acids. A polypeptide reagent according to the invention may be labelled or otherwise, according to the diagnostic method used. Diagnostic methods are described earlier in the text.

According to another aspect, the invention provides a monospecific antibody capable of recognizing a protein or a polypeptide according to the invention. “Monospecific antibody” is understood to mean an antibody capable of reacting predominantly with a single Helicobacter protein. Such an antibody can only be obtained using a substantially purified protein as immunogen. An antibody according to the invention may be polyclonal or monoclonal; the monoclonals may be chimeric (for example, consisting of a variable region of murine origin associated with a human constant region) or humanized (only the hypervariable regions are of animal origin, for example of murine origin) and/or a single chain. The polyclonals, like the monoclonals, may also be in the form of immunoglobulin fragments, for example an F(ab)′ 2 or Fab fragment. An antibody according to the invention may also be of any isotype, for example IgG or IgA; a polyclonal may be of a single isotype or a mixture of all or some of them.

In the text which follows, the terms “monospecific antibody” and “monospecific antiserum” are used interchangeably.

An antibody which is directed against a protein according to the invention may be produced and subsequently identified using a standard immunological assay, for example Western blot, dot blot or ELISA analysis (see for example Coligan et al., Current Protocols in Immunology (1994) John Wiley & sons Inc., New York, N.Y.); Antibodies:A laboratory Manual, D. Lane, (1988) Harlow Ed.).

An antibody according to the invention may be useful in diagnosis, as well as in affinity chromatography for large-scale purification of a protein or a polypeptide according to the invention; such an antibody is also potentially useful as therapeutic agent in a passive immunization procedure.

Consequently, the invention also provides (i) a reagent for detecting the presence of Helicobacter in a biological sample, which comprises an antibody or a polypeptide according to the invention; and (ii) a diagnostic method for detecting the presence of Helicobacter in a biological sample, according to which the biological sample is brought into contact with an antibody or a polypeptide according to the invention, so that an immune complex forms; optionally, the unbound material is removed and the immune complex formed between the sample and the antibody or the polypeptide according to the invention is detected as an indicator of the presence of Helicobacter in the sample or in the organ from which the sample was collected.

As can be easily understood, an antibody according to the invention makes it possible to test for the presence of Helicobacter in a gastric extract.

For use in a diagnostic test, the reagent for to be [sic] be provided in a free form or may be immobilized on a solid support; the latter may be any support commonly used in this domain, for example a tube, a bead or a well. The immobilization may be obtained by direct or indirect means. The direct means comprise passive adsorption (noncovalent bonding) or covalent bonds between the support and the reagent. “Indirect means” means that an anti-reagent compound capable of interacting with a reagent is first attached to a solid support. For example, if a polypeptide reagent is used, an antibody capable of binding it may be used as anti-reagent, provided that it can bind to an epitope of the polypeptide which is not involved in the recognition of the antibodies present in the biological samples. Indirect means may also be implemented through a ligand-receptor system, for example by grafting a molecule, such as a vitamin, onto a polypeptide reagent and then by immobilizing, in solid form, the corresponding receptor. This is illustrated e.g. by the biotin-streptavidin system. Alternatively, indirect means are used, for example, by adding a peptide tail to the reagent, e.g. by chemical means, and by immobilizing the grafted product by passive adsorption or by covalent bonding of the peptide tail.

The invention also relates to a process for the purification of a protein or of a polypeptide according to the invention from a biological sample, according to which the biological sample is subjected to an affinity chromatography using a monospecific antibody according to the invention.

To this end, the antibody may be polyclonal or monoclonal, preferably of the IgG type. Purified IgGs may be prepared from an antiserum according to methods which are commonly used (see for example Coligan et al.).

Conventional chromatography supports, like standard methods of grafting antibodies, are described for example in: Antibodies:A Laboratory Manual, D. Lane, Harlow Ed. (1988).

Briefly, a biological sample, preferably a buffer solution, is applied to a chromatography material, preferably equilibrated with the buffer used for the dilution of the biological sample so that the protein or the polypeptide according to the invention (antigen) can be adsorbed onto the material. The chromatography material, such as a gel or a resin associated with an antibody according to the invention, may be provided in the form of a bath or a column. The components which remain unbound are removed by washing and the antigen is then eluted in an appropriate elution buffer, such as for example a glycine buffer or a buffer containing a chaotropic agent, e.g. guanidine-HCl, or a salt-rich concentration (for example 3 M MgCl₂). The eluted fractions are recovered and the presence of the antigen is then detected, for example, by measuring the absorbance at 280 nm.

Such a purification process may, for example, be used to purify a protein from a total extract. However, if the antibody is not perfectly monospecific, it is advisable to enrich beforehand the material intended to be subjected to the immunoaffinity chromatography in terms of quantity of protein to be purified. For example, such a process may be used to perfect the purification of the 32 kDa protein as obtained according to the process described above which comprises a step of purification on SP-Sepharose.

The therapeutic or prophylactic usefulness of an antibody according to the invention may be demonstrated according to the protection test by Lee et al., proposed above for the proteins or polypeptides according to the invention. Thus, the subject of the invention is also (i) a composition of material comprising a monospecific antibody according to the invention, and a diluent or a carrier; in particular, (ii) a pharmaceutical composition comprising a monospecific antibody according to the invention in an effective quantity from a therapeutic or prophylactic point of view; (iii) the use of a monospecific antibody according to the invention in the preparation of a medicament for treating or preventing a Helicobacter infection; as well as (iv) a method for treating or preventing a Helicobacter infection (for example, H. pylori, H. felis, H. mustelae or H. heilmanii), according to which a therapeutically or prophylactically effective quantity of an antibody according to the invention is administered to an individual requiring such a treatment.

To this end, the monospecific antibody may be polyclonal or monoclonal, preferably of IgA isotype (predominantly). In the context of a passive immunization method, the antibody is administered by the mucosal route to a mammal, for example at the level of the gastric mucous membrane, either by the oral or intragastric route, advantageously in the presence of a bicarbonate buffer. A monospecific antibody according to the invention may be administered as sole active component or as a mixture comprising at least one monospecific antibody specific to each Helicobacter polypeptide. The dose of antibody which should be used in this method can be easily determined by persons skilled in the art. For example, it is suggested that a dosage may be characterized by a daily administration of between 100 and 1000 mg of antibody for one week, or a dose comprising 100 to 1000 mg of antibody administered three times per day for two to three days.

A pharmaceutical composition comprising an antibody according to the invention may be manufactured according to the rules stated above for a composition comprising a protein or a polypeptide according to the invention. Likewise, identical medical indications apply.

The invention is illustrated below with reference to the following Figures:

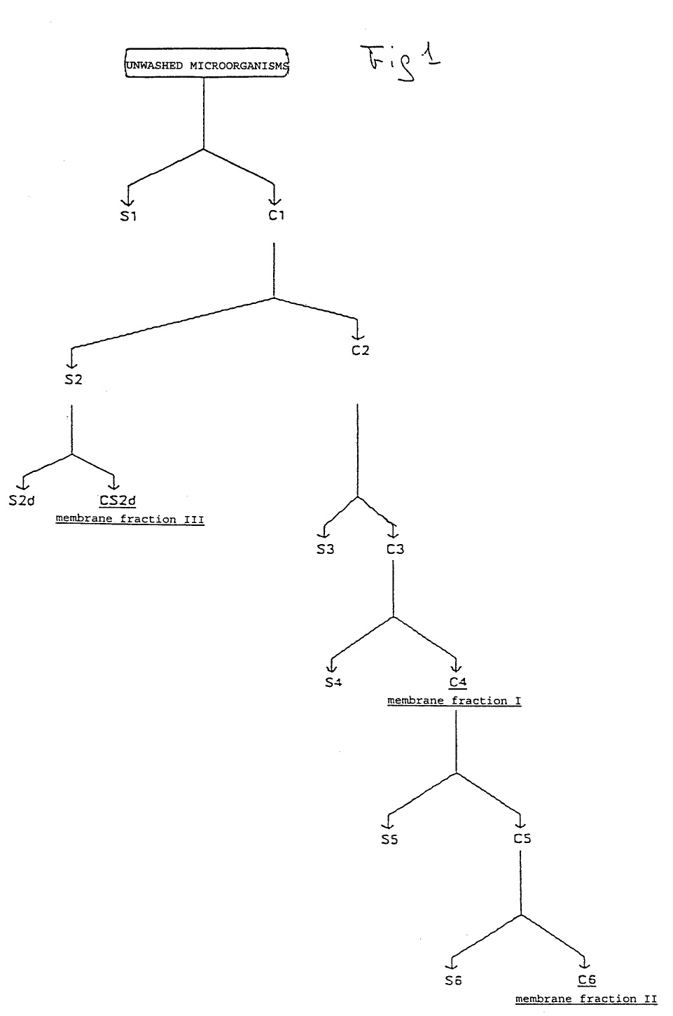

FIG. 1 is a summary of the procedure for the preparation of the H. pylori membrane fractions I, II and III.

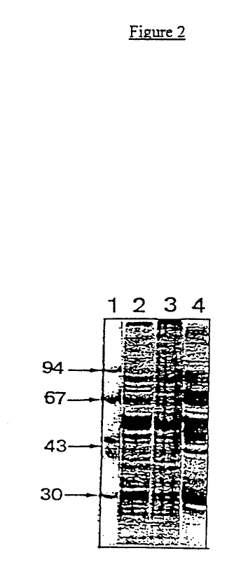

FIG. 2 presents the analysis of the membrane fractions I, II and III by electrophoresis on a 10% polyacrylamide gel and staining with Coomassie blue. The samples loaded are: membrane fraction I (lane 2), membrane fraction II (lane 3), membrane fraction III (lane 4) and molecular weight markers (lane 1).

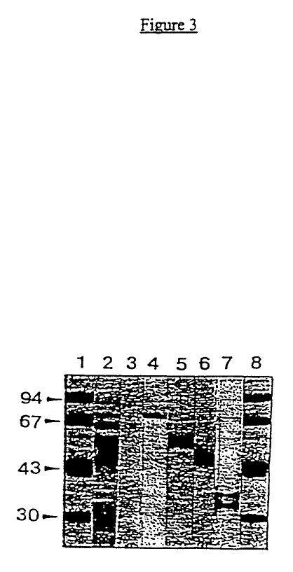

FIG. 3 presents the analysis, by electrophoresis on a 10% polyacrylamide gel and staining with Coomassie blue, of the proteins purified from a preparative gel (lanes 3 to 7). The samples loaded are: the HpP1 fraction (lane 3), the HpP2 fraction (lane 4), the HpP4 fraction (lane 5), the HpP5 fraction (lane 6), the HpP6 fraction (lane 7), the molecular weight markers (lanes 1 and 8) and the membrane fraction I (lane 2).

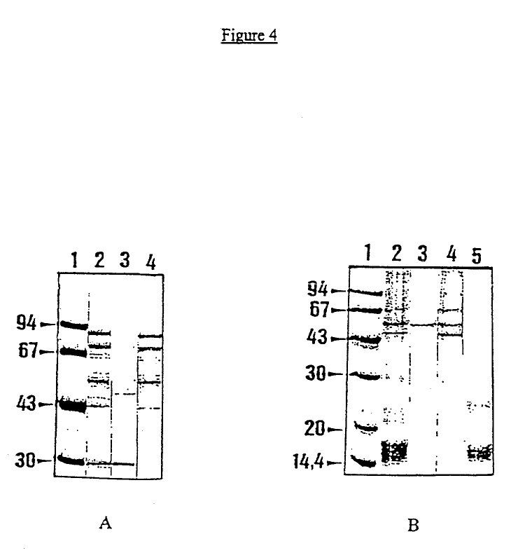

FIG. 4 represents the analysis of the fractions obtained from the passage on DEAE-Sepharose of the fractions 7 and 9 (obtained after elution on Q-Sepharose). The fractions were separated by electrophoresis on a 10% or 12.5% polyacrylamide gel and stained with Coomassie blue. The samples loaded are: fraction 7 (lane 2A), fraction 7.1 (lane 3A), fraction 7.2 (lane 4A), fraction 9 (lane 2B), fraction 9.1 (lane 3B), fraction 9.2 (lane 4B), fraction 9.3 (lane 5B) and molecular weight markers (kDa) (lane 1A and 1B).

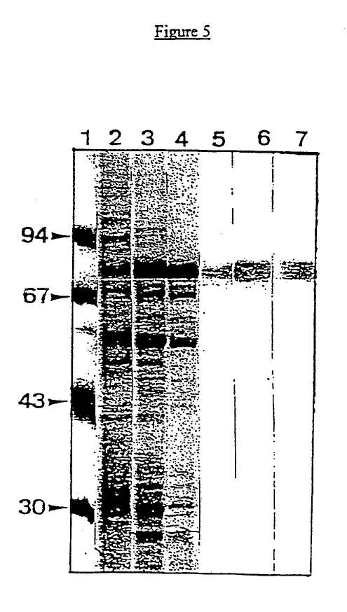

FIG. 5 presents, after electrophoresis on a 10% polyacrylamide gel and staining with Coomassie blue, the electrophoretic profile of the D fraction obtained from the chromatography on a Q-Sepharose column of the membrane fraction III (lane 3) and of the fraction D′ obtained from the chromatography on an S-Sepharose column of the fraction D (lane 4). Lane 1 corresponds to the molecular weight markers and lane 2 to the membrane fraction III.

### EXAMPLE 1

### Preparation of the Membrane Fractions

### 1A—Culture The H. pylori strain ATCC 43579 is cultured in liquid medium in a 10 fermenter.

A frozen sample of microorganisms in glycerol is used to inoculate a 75-cm² flask containing a so-called “two-phase” medium (a solid phase in Colombia agar containing 6% fresh sheep blood and a liquid phase in soya bean trypcase containing 20% foetal calf serum). After 24 hours of culture under microaerophilic conditions, the liquid phase of this culture is used to inoculate several 75-cm² flasks in a two-phase medium in the absence of sheep blood. After 24 hours of culture, the liquid phase makes it possible to inoculate a 2-1biofermenter in liquid soya bean trypcase medium containing beta-cyclodextrin at 10 g/l. This culture at OD 1.5-1.8 is inoculated into a 10-l fermenter in liquid medium. After 24 hours of culture, the bacteria are harvested by centrifugation at 4000×g for 30 minutes at 4° C. A 10-liter culture of. H. pylori ATCC 43579 in a fermenter makes it possible to obtain about 20 to 30 g (wet weight) of bacteria.

### 1B—Extraction with n-octyl β-D-glucopyranoside (OG)

The pellet of microorganisms which is obtained above is washed with 500 ml of PBS (phosphate buffered saline; NaCl 7.650 g, disodium phosphate 0.724 g, monopotassium phosphate 0.210 g per liter; pH 7.2) per liter of culture. The microorganisms are then centrifuged again under the same conditions.

The bacterial pellet obtained (C₁) is resuspended in an OG solution (Sigma) at 1% (30 ml/liter of culture). The bacterial suspension is incubated for 1 hour at room temperature, with magnetic stirring, and then centrifuged at 17,600×g for 30 minutes at 4° C.

The pellet (C₂) is stored for subsequent treatment.

The supernatant (S₂) obtained is dialysed (MWCO=10000 Da, Spectra/por) overnight at 4° C., with magnetic stirring, against twice 1 liter of PBS diluted ½. The precipitate formed during the dialysis is recovered by centrifugation at 2600×g for 30 minutes at 4° C. The supernatant (S_{2d}) is removed and the pellet (C_{s2d}) which contains membrane proteins is stored at −20° C.

### 1C—Breaking of the Microorganisms

The pellet (C₂) obtained after centrifugation of the microorganisms treated with OG is resuspended in 20 mM tris-HCl buffer pH 7.5 and 100 μM Pefabloc (buffer A) and then homogenized by Ultra-turrax (3821, Janke & Kungel). The homogenate obtained is exposed to lysozyme (0.1 mg/ml final) and EDTA (1 mM final).

The homogenate is sonicated for 3 times 2 minutes at 40° C. (probeφ=0.5 cm, power =20%, Sonifier 450 Branson), and then ultracentrifuged at 210,000×g for 30 minutes at 40° C. The supernatant (S₃) which contains cytoplasmic and periplasmic proteins is removed, while the pellet (C₃) is recovered, washed with buffer A, and then ultracentrifuged at 210,000×g for 30 minutes at 40° C. After removal of the supernatant (S₄), the pellet (C₄) is stored at −20° C. This pellet contains intrinsic and peripheral membrane proteins.

The procedure may be continued by a double washing of the pellet C₄ in order to remove peripheral membrane proteins. The pellet C₄ is resuspended in 50 mM NaCO₃ buffer pH 9.5, 100 μM Pefabloc (buffer B). The suspension is ultracentrifuged at 210,000×g for 30 minutes at 40° C. The supernatant (S₅) is removed and then the pellet (C₅) is washed and ultracentrifuged under the same conditions as above. After removal of the supernatant (S₆), the pellet (C₆) which contains essentially intrinsic membrane proteins is stored at −20° C.

The fractions C4, C6 and C_{s2d} are called hereinafter membrane fractions I, II and III respectively.

### 1D—Analysis of the Membrane Fractions

The various membrane fractions are analysed by polyacrylamide gel electrophoresis in the presence of SDS according to the Laemmli method (1970). The proteins are visualized after Coomassie blue staining.

If the major proteins of each fraction are considered, the SDS-PAGE profiles (FIG. 2) show that the membrane fraction I is very similar to the membrane fraction II. On the other hand, these two differ substantially from membrane fraction III.

The profile of membrane fraction I shows 7 major protein bands of respective molecular weights 87, 76, 67, 54, 50, 47 and 32-35 kDa (lane 2). By Western blotting in the presence of anti-ureB antibody or anti-catalase antibody, it was shown that the band at 67 kDa corresponded to the B subunit of urease and the band at 54 kD corresponded to catalase. These two proteins do not exist in the profile of fraction II (lane 3) since the washing with carbonate buffer removes the proteins weakly associated with the membrane.

As for the protein profile of the membrane fraction III, it shows the presence of 4 major bands at 76, 67, 50 and 30 kDa (lane 4).

### EXAMPLE 2

### Purification of the Proteins of the Membrane Fraction I by Preparative SDS-PAGE

An electrophoresis is carried out on polyacrylamide gel according to the Laemmli method (1970) with a 5% stacking gel and a 10% separating gel. The membrane fraction is resuspended in buffer A and then diluted one half in 2× sample buffer. The mixture is heated for 5 minutes at 95° C. About 19 mg of proteins are loaded onto a gel 16×12 cm in size and 5 mm thick. A premigration is performed at 50 V for 2 hours, followed by a migration at 65 V overnight. The staining of the gel with Coomassie blue R250 (0.05% in ultrafiltered water) allows good visualization of the bands.

The major bands HpP1, HpP2, HpP4, HpP5 and HpP6 are cut out with a scalpel and ground in an Ultra-turrax in the presence of 10 or 20 ml of extraction buffer containing 25 mM Tris-HCl pH 8.8, 8 M urea, 10% SDS, 100 μM phenylmethylsulphonyl fluoric [sic] (PMSF) and 100 μM Pefabloc (buffer C). Each ground product is filtered on a Millipore AP20 prefilter (φ_{filter}=4-7 cm, φₚₒᵣₑ=20 μm) with the aid of an extruder at a pressure of 7 bar, at room temperature. Each ground product is washed with 5 to 10 ml of buffer C and filtered as above. The two filtrates obtained from each corresponding ground product are combined.

Each filtrate is precipitated with 3 volumes of a 50:50 mixture 75% methanol and 75% isopropanol, and then ultracentrifuged at 240,000×g for 16 hours at 10° C. on a 70 TFT rotor (J8-55, Beckman).

Each pellet is taken up in 2 ml. of solubilization buffer containing 10 mM NaPO₄ pH 7.0, 1 M NaCl, 0.1% sarcosyl, 1.00 μM PMSF, 100 μM Pefabloc and 6 M urea (buffer D). The solubilized sample is dialysed successively against 100 ml of buffer D containing 4 M urea and 0.1% sarcosyl, against 100 ml of buffer D containing 2 M urea and 0.5% sarcosyl and against twice 100 ml of buffer D without urea and containing 0.5% sarcosyl. The dialysis is carried out for 1 hour, with magnetic stirring, at room temperature. The final dialysate is incubated for 30 minutes in an ice bath and then centrifuged at low speed for 10 minutes at 4° C. (Biofuge A, Heraeus Sepatech). The supernatant is recovered, filtered on a 0.45 μm Millipore filter and stored at −20° C.

An SDS-PAGE analysis was carried out for each fraction (FIG. 3).

Analysis of the electrophoretic profile of each fraction shows that the fractions HpP1, HpP2 and HpP4 are pure with a single gel band for each of these fractions (at 87, 76 and 54 kDa respectively). The fraction HpP5 has a band of high intensity at 50 kDa which is slightly contaminated with a band at 47 kDa; likewise the fraction HpP6 has a band of high intensity at 32 kDa which is slightly contaminated with a band at 35 kDa.

### EXAMPLE 3

### Purification of the Membrane Proteins of 30, 50 and 54 kD from the Membrane Fraction I

### 5 3A—Anion-exchange Chromatography on Q-Sepharose

A Q-Sepharose column of 40 ml (φ=2.5 cm, h=8 cm) is prepared according to the recommendations of the manufacturer (Pharmacia). The column is washed and then equilibrated with the 50 mM NaCO₃ buffer pH 9.5 containing 100 μM Pefabloc and 0.1% zwittergent 3-14. The chromatography was monitored by UV detection at 280 nm at the outlet of the column.

One hundred and forty mg of previously solubilized proteins of membrane fraction I are loaded onto the column which is then washed with the equilibrating buffer (50 mM NaCO₃ pH 9.5, 100 μM Pefabloc and 0.1% zwittergent 3-14) until the absorbance at 280 nm is stabilized. The proteins are eluted by a 0.1 to 0.5 M NaCl gradient in the equilibration buffer (10 times V_{T}) followed by washing in equilibration buffer containing 0.5 and 1 M NaCl (twice V_{T}). The fractions collected are analysed by SDS-PAGE and 25 combined into different pools according to their electrophoretic profile, and then stored at −20° C.

The fractions are as follows:

| Fractions | Elution | Fractions | Elution |
|---|---|---|---|
| 1 | direct eluate | 6 | 0.25-0.28M |
| | | | NaCl |
| 2 | washing | 7 | 0.28-0.35M |
| | equilibration | | NaCl |
| | buffer | | |
| 3 | 0-0.1M NaCl | 8 | 0.35-0.5M |
| | | | NaCl |
| 4 | 0.1-0.2M | 9 | start of |
| | NaCl | | washing 1M |
| | | | NaCl |
| 5 | 0.2-0.25M | 10 | end of washing |
| | NaCl | | 1M NaCl |

The protein evaluation shows that 53% of the proteins are eluted in the 0-0.5 M NaCl gradient, 14% of the proteins are not attached to the column and 33% of the proteins are eluted during the washing in 1 M NaCl (Table 5). The proteins which are not bound to the column correspond to alkaline proteins which are positively charged at pH 7.5, whereas the proteins eluted in 1 M NaCl correspond to acidic proteins which are highly charged at this pH.

The purification of the fractions 7 and 9 is continued as follows.

### 3B—Separation of the Proteins of Fractions 7 and 9 by Anion-exchange Chromatography on DEAE-Sepharose

A DEAE-Sepharose column is prepared according to the recommendations of the manufacturer (Pharmacia) for a gel volume of about 10 ml (φ=1.5 cm, h=5 cm) (maximum 10 mg protein/ml of gel). The column is washed and then equilibrated with the 50 mM Tris-HCl buffer pH 7.5 containing 100 μA Pefabloc and 0.1% zwittergent 3-14. The chromatography is monitored as before by UV detection at 280 nm at the outlet of the column.

The fraction 7 dialysed beforehand against the equilibration buffer (50 mM Tris-HCl pH 7.5, 100 μM Pefabloc and 0.1% zwittergent 3-14) containing 10 mg of proteins is loaded onto the DEAE-Sepharose column. The column is washed with equilibration buffer until the absorbance at 280 nm is stabilized. The proteins are eluted by a 0 to 0.5 M NaCl gradient in the equilibration buffer (10 times V_{T}), followed by washing with equilibration buffer containing 1 M NaCl (twice VT). The fractions collected are analysed by SDS-PAGE and then combined into different pools according to their protein profile and stored at −20° C. By SDS-PAGE, it is shown that the fraction 7.1 (direct eluate) is of interest.

An identical purification is repeated with the fraction 9 containing 31 mg of protein. By SDS-PAGE, it is shown that the fractions 9.1, 9.2 and 9.3 eluted at 0.1-0.25 M NaCl, 0.3-0.4 M NaCl and 1 M NaCl, respectively, are of interest.

For the fraction 7 (FIG. 4A), the results obtained show that only a protein of 30 kDa (lane 3; fraction 7.1) was enriched and partially separated after passage through the DEAE-Sepharose column, the other proteins were not separated. For the fraction 9 (FIG. 4B), the electrophoretic profiles show that two proteins of 54 and 15 kDa (lanes 3 and 5; fractions 9.1 and 9.3) were separated and a protein of 50 kDa was enriched (lane 4; fraction 9.2). The protein of 54 kDa of fraction 9.1 does not react with anti-catalase antibodies.

### EXAMPLE 4

### Purification of the Membrane Protein of 32 kDa from the Membrane Fraction I

The membrane fraction I is solubilized in 50 mM NaCO₃ buffer pH 9.5 at room temperature for 30 min, with stirring. The suspension is then centrifuged at 200,000×g for 30 min at +40° C. The supernatant is dialysed against 50 mM NaPO₄ buffer pH 7.4 and then loaded onto an SP-Sepharose column previously equilibrated with this same buffer. After washing the column with this same buffer, the column is subjected to a 0-0.5 M NaCl gradient. The fraction eluted between 0.26 and 0.31 M contains the protein of 32 kDa.

### EXAMPLE 5

### Preparation of Hyperimmune Sera Against the Fractions HpP5 and HpP6.

Polyconal sera specific for the H. pylori major membrane proteins are obtained by hyperimmunization of rabbits respectively with the antigens purified by preparative SDS-PAGE HpP5 and HpP6. The first injection DO (subcutaneous multisite and intramuscular) is carried out with a preparation containing 50 μg of emulsified membrane protein in complete Freund's adjuvant, and then the boosters D21 and D42 are made by injection of 25 μg of membrane protein in incomplete Freund's adjuvant. The animals are sacrificed on D60. The sera obtained are decomplementized for 30 minutes at 56° C. and sterilized by filtration on a membrane with a porosity of 0.22 μm (Millipore).

The anti-HpP5 antiserum reacts with the 50 kDa protein isolated in the fraction 9.2 obtained in Example 3. The anti-HpP6 antiserum reacts with the 32 kDa protein isolated in the fraction eluted between 0.26-0.31 M NaCl on SP-Sepharose, as obtained in Example 4.

Quite obviously, the immunization procedure described above may be used in a similar manner to produce antisera against each of the proteins purified in Example 3. It will be possible for the preparations obtained in these Examples to be advantageously subjected to a preparative electrophoresis on an SDS-PAGE gel. The protein bands will be treated as above so as to obtain a preparation intended for immunization.

### EXAMPLE 6

### Purification of a Catalase from H. pylori

A culture is performed as described in Example 1A. The washed bacterial pellet is resuspended in 50 mM sodium phosphate buffer pH 7.5 containing 100 μM PMSF (phenylmethylsulphonyl fluoride Sigma) (buffer A) at a final concentration of 0.1 g (wet weight) per milliliter. The suspension is homogenized with the aid of an Ultraturrax-type mixer. The bacterial cells are then broken by sonication with a Sonifier-type apparatus (Branson) equipped with a probe with a diameter of 1.8 cm. The sonication is performed intermittently, 1 min of sonication and 1 min of rest on ice. A 10-min sonication is sufficient to break completely 5 g of microorganisms in suspension. The lysate thus obtained is centrifuged for 15 min at 40° C. at 4000 g. The supernatant is recovered and then again centrifuged at 100,000 g for 30 min at 40° C. The supernatant from this second centrifugation (S2) is recovered for chromatographic purification. The fraction S2 prepared in this manner retains about 90% of the total “catalase” enzymatic activity, as measured according to the Hazell et al. (supra) technique or Beers & Sizer, J. Biol. Chem. (1952) 195:133 technique.

The fraction S2 is loaded onto an S-Sepharose column (Pharmacia) previously equilibrated with buffer A. The column is washed with the same buffer. The chromatography is monitored with a UV detector at 280 nm for the proteins and by the enzymatic activity for the catalase. After removal of the unbound proteins (absorption at 280 nm returned to the baseline), the column is then washed with a 0 to 1 M NaCl gradient in buffer A. The fractions corresponding to the catalase activity peak are recovered, concentrated in an Amicon-type concentration cell equipped with a membrane whose molecular weight cut-off is 100,000 Daltons. The concentrated fraction thus obtained is loaded onto a Sephacryl S-300 HR column previously equilibrated with PBS buffer. The fractions containing the catalase activity are combined, concentrated to 1 mg/ml and dialysed against the PBS buffer. The final solution is filtered on a membrane with a porosity of 0.22 μm and stored at −70° C.

The protein thus purified has the following characteristics:

(i) A typical catalase enzymatic activity, in the absence of peroxidase activity.

(ii) A visible spectrum typical of a haemoprotein, a Soret peak at 406 nm and alpha and beta peaks between 520 nm and 550 nm.

(iii) A monomeric form at 54 kDa in SDS-PAGE with the following N-terminal sequence: MVNKDVKQTTAFGAPVWDDNNVITAGPRG.

### EXAMPLE 7

### Purification of the Membrane Protein of 50 kDa by Immunoaffinity

### 7.A—Purification of the IgGs

A hyperimmune serum against fraction HpP5 as prepared in Example 5 is loaded onto a Protein A Sepharose 4 Fast Flow column (Pharmacia) previously equilibrated in 100 mM Tris-HCl pH 8.0. The resin is washed with 10 column volumes of 100 mM Tris-HCl pH 8.0 and then with 10 column volumes of 10 mM Tris-HCl pH 8.0. The IgGs are eluted in 0.1 M glycine buffer pH 3.0. The IgGs are collected as 5-ml fractions to which 0.25 ml of 1 M Tris-HCl pH 8.0 is added. The optical density of the eluate is measured at 280 nm and the fractions containing the IgGs are combined and, if necessary, frozen at −70° C.

### 7.B—Preparation of the Column

An appropriate quantity of CNBr-activated Sepharose 4B gel (knowing that 1 g of dry gel gives about 3.5 ml of hydrated gel and that the capacity of the gel is 5 to 10 mg of IgG per ml of gel) manufactured by Pharmacia (ref: 17-0430-01) is suspended in 1 mM NaCl buffer. The gel is then washed with the aid of a buchner by adding small quantities of 1 mM HCl. The total volume of 1 mM HCl used is 200 ml per gram of gel.

The purified IgGs are dialysed for 4 h at 20 +50° C. against 50 vol. of 500 mM sodium phosphate buffer pH 7.5. They are then diluted in 500 mM sodium phosphate buffer pH 7.5 to a final concentration of 3 mg/ml.

The IgGs are incubated with the gel overnight at 5+3° C., with rotary stirring. The gel is placed in a chromatography column and washed with 2 column vol. of 500 mM phosphate buffer pH 7.5. The gel is then transferred into a tube and incubated in 100 mM ethanolamine pH 7.5 at room temperature, with stirring. It is then washed with 2 column vol. of PBS. The gel may be stored in PBS merthiolate 1/10,000. The quantity of IgGs coupled to the gel may be determined by measuring the difference in optical density at 280 nm between the initial IgG solution and the direct eluate plus the washings.

### 7.C—Adsorption and Elution of the Antigen

A protein preparation of antigen in 50 mM Tris-HCl pH 8.0, 2 mM EDTA, for example the membrane fraction I or II (fraction C4 or C6 as obtained in Example 1C and solubilized in zwittergent) is filtered through a 0.45 μm membrane and is then loaded onto the column previously equilibrated with 50 mM Tris-HCl pH 8.0, 2 mM EDTA, at a flow rate of about 10 ml/h. The column is then washed with 20 vol. of 50 mM Tris-HCl pH 8.0, 2 mM EDTA. Alternatively, the adsorption may take place in a bath; the incubation is continued at 5+3° C. overnight and with stirring.

The gel is washed with 2 to 6 vol. of 10 mM sodium phosphate buffer pH 6.8. The antigen is eluted with 100 mM glycine buffer pH 2.5. The eluate is harvested in 3-ml fractions to which 150 μl of 1 M sodium phosphate buffer pH 8.0 are added. The optical density of each fraction is measured at 280 nm; the fractions containing the antigen are combined and stored at −700° C. The analysis by electrophoresis on a 10% SDS-Page gel shows only one band at 50 kDa.

### EXAMPLE 8

### Purification of the Membrane Protein of 32 kDa by Immunoaffinity

Example 7 is repeated using the antiserum against fraction HpP6, in order to continue the purification of the fraction eluted between 0.26 and 0.31 M NaCl as described in Example 4. The fractions collected after elution and containing the protein are combined into a single preparation; the latter is analysed by SDS-Page electrophoresis on a 10% gel. A single band appears at 32 kDa.

### EXAMPLE 9

### Agglutination Test

### 9.A—Culture

From a strain of H. pylori No. ATCC 43579 (available from ATCC, 12301 Parklawn Drive, Rockville Md.—USA) stored in glycerol at −70° C., a 25 cm² flask containing a two-phase medium is inoculated. The two-phase medium comprises a solid phase consisting of 10 ml of Colombia agar (BioMerieux) supplemented with 6% fresh sheep blood and a liquid phase consisting of 3 ml of soya bean Trypcase broth (Difco) containing 20% foetal calf serum. The flasks are placed in a sealed bag called “generbag” (BBL) and incubated with gentle rotary shaking at 37° C. for 48 hours under microaerophilic condition (8-10% CO₂, 5-7% O₂ and 85-87% N₂) obtained by the Microaer System (BBL).

This 48-hour culture is used to again inoculate flasks containing two-phase medium. The initial absorbance of this culture at 600 nm should be between 0.15 and 0.2. The flasks are incubated under conditions identical to those described above.

After 48 hours, the bacterial suspension is transferred to a test tube. The absorbance of this culture is measured and it should be between 3.0 and 3.5 at 600 nm. The appearance of the microorganisms is checked under a microscope after Gram staining.

### 9.B—Antisera

An antiserum as obtained in Example 5 is filtered on a 0.45 μm membrane so as to remove small aggregates, if they exist, before use.

### 9.C—Agglutination Test

On a black-bottomed immunoprecipitation plate (Prolabo ref. 10050), there are deposited 20 μl of physiological saline in the first well, 20 μl of serum, collected before immunization, in the central well and 20 μl of antiserum in the third well. Twenty μl of bacterial suspension of H. pylori are added to each of the three wells and the drops are then mixed with the aid of a Pasteur pipette with a sealed round tip.

The onset of agglutination is observed under a magnifying glass at most 5 minutes after the mixing. The agglutination is complete when the mixture appears in the form of a clear solution comprising large aggregates. The negative controls, either with physiological saline, or with the preimmunization serum, should remain cloudy, revealing that the bacterial suspension is intact.

The antiserum against fraction HpP5 and against fraction HpP6 give a very strong agglutination reaction. Under the conditions tested, the H. pylori bacteria agglutinate rapidly and the reaction is complete after one minute. The results indicate that the proteins of 50 and 32 kDa are probably exposed at the surface of H. pylori.

### EXAMPLE 10

### Demonstration of the Protective Effect of the Membrane Proteins of 54, 50, 30 and 32 kDa

Groups of about ten Swiss Webster mice aged from 6 to 8 weeks (Taconic Labs, Germantown, N.Y.) are immunized by the intragastric route with 1, 5, 25, 50 or 100 μg of the antigen of 54, 50 or 30 kDa purified by chromatography as described in Example 3, or of the antigen of 32 kDa purified by chromatography as described in Example 4 or by immunoaffinity as described in Example 8, or of the antigen of 50 kDa purified by immunoaffinity as described in Example 7 (preferred). The antigen is diluted in PBS or in PBS containing 0.24 M sodium bicarbonate. The antigen is supplemented with 5 or 10 μg of cholera toxin (CT) (Calbiochem, San Diego) or with heat-labile toxin (LT) (Berna Products, Coral Gables Fla.). The mice are first anaesthetized with isoflurane and then the dose is administered in a volume of about 0.5 ml with the aid of a cannula. Four doses are administered to each mouse at 7-10 day intervals. Two weeks after the last administration of antigen, the mice are challenged with a single dose of H. pylori ORV2002 strain (1×10⁷ live bacteria in 200 μl of PBS; OD₅₅₀ of about 0.5) administered by the intragastric route. A group having received no dose of antigen and serving as control is challenged likewise. Two weeks after the challenge, the mice are sacrificed. The percentage of protection is determined either by measuring the urease activity or by evaluating the bacterial load by histology as described in Lee et al. (supra) or directly by quantitative culture of H. pylori. Under these conditions, it is possible to observe for each of the proteins of 54, 50, 30 and 32 kDa, a substantial reduction in the infectious load in most of the mice immunized with 25 μg compared with the control group; this makes it possible to conclude that the H. pylori antigens of 54, 50, 30 and 32 kDa are at least partially protective; the best results being obtained with the protein of 32 kDa (100% protection).

1. Helicobacter pylori protein in a substantially purified form, capable of being obtained from an H. pylori membrane fraction, and whose molecular weight after electrophoresis on a 10% polyacrylamide gel in the presence of SDS appears of the order of 54, 50, 32-35 or 30 kDa; provided that when the molecular weight is 54 kDa, the protein does not react with an anti-catalase antiserum.

2. Protein according to claim 1, whose apparent molecular weight is of the order of 54 kDa and which is capable of being obtained by a process in which:
(i) the H. pylori bacteria are extracted with 1% n-octyl β-D glucopyranoside, followed by centrifugation;
(ii) a bacterial pellet is recovered and it is treated with lysozyme and subjected to sonication, followed by centrifugation;
(iii) a centrifugation pellet is recovered and it is subjected to washing with 20 mM Tris-HCl buffer pH 7.5, followed by centrifugation;
(iv) the membrane fraction consisting of the centrifugation pellet is recovered and it is resuspended in aqueous medium;
(v) the membrane fraction is subjected to an anion-exchange chromatography on a Q-Sepharose column in a 0-0.5 M NaCl gradient, followed by washing in 1 M NaCl;
(vi) the fraction eluted at the start of washing in 1 M NaCl is recovered and it is subjected to an anion-exchange chromatography on a DEAE-Sepharose column, in a 0-0.5 M NaCl gradient; and
(vii) the fraction eluted in 0.1-0.25 M NaCl is recovered.

3. Protein according to claim 1, whose apparent molecular weight is of the order of 50 kDa and which is capable of being obtained by a process in which:
(i) the H. pylori bacteria are extracted with 1% n-octyl β-D glucopyranoside, followed by centrifugation;
(ii) a bacterial pellet is recovered and it is treated with lysozyme and subjected to sonication, followed by centrifugation;
(iii) a centrifugation pellet is recovered and it is subjected to washing with 20 mM Tris-HCl buffer pH 7.5, followed by centrifugation;
(iv) the membrane fraction consisting of the centrifugation pellet is recovered and it is resuspended in aqueous medium;
(v) the membrane fraction is subjected to an anion-exchange chromatography on a Q-Sepharose column in a 0-0.5 M NaCl gradient, followed by washing in 1 M NaCl;
(vi) the fraction eluted at the start of washing in 1 M NaCl is recovered and it is subjected to an anion-exchange chromatography on a DEAE-Sepharose column, in a 0-0.5 M NaCl gradient; and
(vii) the fraction eluted in 0.3-0.4 M NaCl is recovered.

4. Protein according to claim 3, which has as N-terminal sequence the amino acid sequence as shown in SEQ ID NO 1.

5. Protein according to claim 1, whose apparent molecular weight is of the order of 30 kDa and which is capable of being obtained by a process in which:
(i) the H. pylori bacteria are extracted with 1% n-octyl β-D glucopyranoside, followed by centrifugation;
(ii) a bacterial pellet is recovered and it is treated with lysozyme and subjected to sonication, followed by centrifugation;
(iii) a centrifugation pellet is recovered and it is subjected to washing with 20 mM Tris-HCl buffer pH 7.5, followed by centrifugation;
(iv) the membrane fraction consisting of the centrifugation pellet is recovered and it is resuspended in aqueous medium;
(v) the membrane fraction is subjected to an anion-exchange chromatography on a Q-Sepharose column in a 0-0.5 M NaCl gradient;
(vi) the fraction eluted in 0.28-0.35 M NaCl is recovered and it is subjected to an anion-exchange chromatography on a DEAE-Sepharose column, in a 0-0.5 M NaCl gradient; and
(vii) the fraction corresponding to the direct eluate is recovered (absence of NaCl).

6. Protein according to claim 1, whose apparent molecular weight is of the order of 32-35 kDa and which is capable of being obtained by a process in which:
(i) the H. pylori bacteria are extracted with 1% n-octyl β-D glucopyranoside, followed by centrifugation;
(ii) a bacterial pellet is recovered and it is treated with lysozyme and subjected to sonication, followed by centrifugation;
(iii) a centrifugation pellet is recovered and it is subjected to washing with 20 mM Tris-HCl buffer pH 7.5, followed by centrifugation;
(iv) the membrane fraction consisting of the centrifugation pellet is recovered and it is resuspended in aqueous medium, advantageously in carbonate buffer pH 9.5;
(v) the suspension obtained in (iv) is centrifuged at about 200,000×g and the supernatant is recovered;
(vi) the pH of the supernatant obtained in (v) is reduced to about pH 7, advantageously by dialysing against phosphate buffer pH 7;
(vii) the preparation obtained in (vi) is subjected to a cation-exchange chromatography on an SP-Sepharose column in a 0-0.5 M NaCl gradient, advantageously in a phosphate buffer pH 7; and
(vii) the fraction eluted in 0.26-0.31 M NaCl is recovered.

7. Helicobacter protein or a polypeptide derived from the protein by fragmentation and/or mutation, in a substantially purified form, which is capable of being recognized by an antiserum raised against a protein according to claim 1.

8. Pharmaceutical composition for the prevention or treatment of an H. pylori infection, which comprises as active ingredient a protein or a polypeptide according to claim 1.

9. Pharmaceutical composition for the prevention or treatment of an H. pylori infection, which comprises as active ingredient a protein or a polypeptide according to claim 7.

10. Monospecific antibody capable of recognizing a protein or a polypeptide according to claim 1.

11. Monospecific antibody capable of recognizing a protein or a polypeptide according to claim 7.

12. Pharmaceutical composition intended for the prevention or treatment of an H. pylori infection, which comprises as active ingredient a monospecific antibody according to claim 10.

13. Pharmaceutical composition intended for the prevention or treatment of an H. pylori infection, which comprises as active ingredient a monospecific antibody according to claim 11.

14. Diagnostic method which makes it possible to detect the presence of Helicobacter in a biological sample, according to which the biological sample is brought into contact with an antibody according to claim 10 so that an immune complex forms, the unbound material is optionally removed and the immune complex formed between the sample and the antibody is detected.

15. Diagnostic method which makes it possible to detect the presence of antibodies to Helicobacter in a biological sample, according to which the biological sample is brought into contact with a polypeptide of claim 7 so that an immune complex forms, the unbound material is optionally removed and the immune complex formed between the sample and the polypeptide is detected.

16. Process for the purification of a protein or of a polypeptide according to claim 1 from a biological sample, according to which the biological sample is subjected to an affinity chromatography using a monospecific antibody according to claim 10.


SEQUENCE LISTING (2 sequences)

SEQ ID NO: 1 - protein, 22 aa, Helicobacter pylori
MKEKFNRTKPHVNIGTIGHVDH

SEQ ID NO: 2 - protein, 29 aa, Helicobacter pylori
MVNKDVKQTTAFGAPVWDDNNVITAGPRG